(19) United States
(12) Patent Application Publication
(10) Pub. No.: US 2024/0182502 A1
(43) Pub. Date: Jun. 6, 2024

(54) METHODS FOR PREPARING L-GLUFOSINATE

(71) Applicant: BASF SE, Ludwigshafen (DE)

(72) Inventors: Klaus Ditrich, Ludwigshafen (DE); Ana Escribano Cuesta, Ludwigshafen (DE)

(21) Appl. No.: 18/284,108

(22) PCT Filed: Mar. 30, 2022
(86) PCT No.: PCT/EP2022/058491

§ 371 Date: Sep. 26, 2023

(30) Foreign Application Priority Data
Apr. 1, 2021 (EP) 21166579.9

(51) Int. Cl. C07F 9/30; C12P 41/00

(52) U.S. Cl. CPC C07F 9/301; C12P 41/006

(57) ABSTRACT

Methods for the production of L-glufosinate (also known as phosphinothricin or (S)-2-amino-4-(hydroxy(methyl)phosphonoyl)butanoic acid) ammonium salt are provided. The methods comprise a refined multi-step process. The first step involves the oxidative deamination of D-glufosinate to PPO (2-oxo-4-(hydroxy(methyl)phosphinoyl)butyric acid). The second step involves the specific amination of PPO to L-glufosinate, using an amine group from one or more amine donors. The third step involves the enrichment of the desired enantiomer in the yield by conversion of the obtained side product to the desired final product as well. By addition of the third refinement step, the proportion of the D-glufosinate present in a mixture of L-glufosinate and D-glufosinate can substantially be converted to the desired L-glufosinate ammonium salt.

### FIELD OF INVENTION

The present invention relates to a method for preparing and obtaining the L-isomer of the herbicide glufosinate. The present invention is suitable for preparing and obtaining the L-isomer out of the racemic mixture of D,L-glufosinate. The present invention is especially suitable for optimizing the yield of obtained L-isomer out of the racemic mixture of D,L-glufosinate.

In addition, the invention is as well generally applicable for methods, which aim to convert alkylammonium salts of acidic compounds into the corresponding ammonium salts.

### BACKGROUND

The herbicide glufosinate, with IUPAC-Name: (2RS)-2-amino-4-[hydroxy(methyl)phosphinoyl]butyric acid or 4-[hydroxy(methyl)phosphinoyl]-DL-homoalanine, CAS Reg. No. 51276-47-2) and with common name DL-4-[hydroxyl(methyl)phosphinoyl]-DL-homoalaninate, is a non-selective, foliar-applied herbicide and is considered to be one of the safest herbicides from a toxicological or environmental standpoint.

In particular glufosinate-ammonium (IUPAC-Name: ammonium (2RS)-2-amino-4-(methylphosphinato)butyric acid, CAS Reg. No. 77182-82-2) is a well known agronomically acceptable salt thereof.

Glufosinate ammonium and its herbicidal acitivity have been described as well e.g. by F. Schwerdtle et al. Z. Pflanzenkr. Pflanzenschutz, 1981, Sonderheft IX, pp. 431-440.

Hoerlein et al., in Rev. Environ. Contam. Toxicol. (Vol. 138, 1994) “Glufosinate (phosphinothricin), a natural amino acid with unexpected herbicidal properties”, discusses the glutamate synthesis inhibitor glufosinate.

U.S. Pat. No. 4,168,963 describes phosphorus-containing compounds with herbicidal activity, of which, in particular, phosphinothricin (2-amino-4-[hydroxy(methyl)phosphinoyl]butanoic acid, with common name: glufosinate) and its salts have acquired commercial importance in the agrochemistry (agricultural chemistry) sector.

Glufosinate is a racemate and represented by the following structure (1):

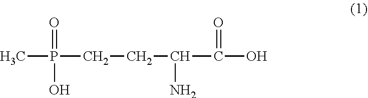

Further glufosinate is a racemate of two enantiomers out of which only one shows sufficient herbicidal activity (see e.g. U.S. Pat. No. 4,265,654 and JP92448/83). L-glufosinate is much more potent than D-glufosinate (Ruhland et al. (2002) Environ. Biosafety Res. 1:29-37).

Current commercial chemical synthesis methods for glufosinate yield a racemic mixture of L-and D-glufosinate (Duke et al. 2010 Toxins 2:1943-1962).

Glufosinate as racemate and its salts are commercially available under the tradenames Basta™ and Liberty™.

L-glufosinate, with IUPAC-Name (2S)-2-amino-4-[hydroxy(methyl)phosphinoyl]butyric acid (CAS Reg. No. 35597-44-5) and also called glufosinate-P, can be obtained commercially or may be prepared for example as described in U.S. Pat. No. 10,260,078 B2, WO2006/104120, U.S. Pat. No. 5,530,142, EP0248357A2, EP0249188A2, EP0344683A2, EP0367145A2, EP0477902A2, EP0127429 and J. Chem. Soc. Perkin Trans. 1, 1992, 1525-1529.

However, enantioselective syntheses based either on conventional, meaning dynamic kinetic resolution of racemates (like in WO18108794), or enzyme catalysed racemate resolution (as with e.g. PG Amidase in EP0054897) or based on asymmetric synthesis such as asymmetric hydrogenations (as in EP0238954 or EP1864989) require additional auxiliaries such as difficult to obtain starting materials or other auxiliaries or require additional synthesis steps rendering the synthesis more complex and thereby costly. Thus, none of these approaches have yet proven to be cost competitive compared to the synthesis of racemic material.

### SUMMARY OF THE INVENTION

Hence in view of the need for the active enantiomer L-glufosinate, methods are still needed, which produce primarily, even preferably solely, the active L-form.

In addition, there is also a need for obtaining in a simple and cost-efficient way especially the ammonium salt of glufosinate, which is a well-established, already registered and agriculturally reliable salt.

Described herein are new and cost-effective methods for the production of L-glufosinate. Especially for obtaining the ammonium salt thereof.

### DETAILED DESCRIPTION

Glufosinate was first synthesized as a racemic mixture. Most of the glufosinate comprising herbicidal products on the market comprise a 50:50 mixture of L-glufosinate and D-glufosinate, whereas the observed herbicidal activity, as mentioned above, is performed by L-glufosinate, while D-glufosinate is not active and instead unleashes chiral herbicidal inactive compounds into the environment.

Hence, there is a need for efficient, simple and cost-effective methods which provide solely, or at least mainly, the active L-isomer of glufosinate.

One method in prior art for obtaining L-glufosinate is through deracemization of D,L glufosinate. For instance, WO 2017151573 describes the deracemization of the racemic glufosinate-ammonium of formula DL-(1) in a two-step process (scheme 1), wherein the first step involves the oxidative deamination of D-glufosinate to PPO (2-oxo-4-(hydroxy(methyl)phosphinoyl)butyric acid) and the second step involves the specific amination of PPO to L-glufosinate ammonium of formula L-(1), using an amine group from one or more amine donors.

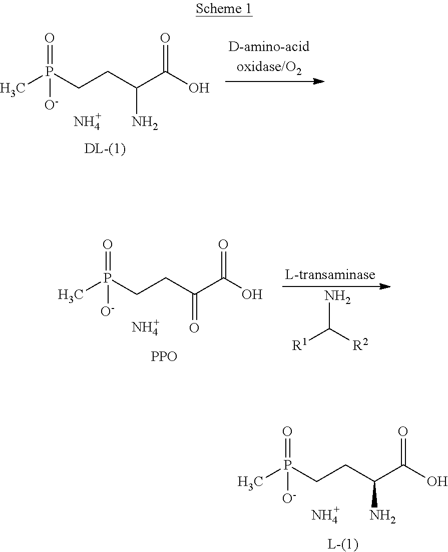

However, although it is said, that by combining these two reactions, the proportion of L-glufosinate could be substantially increased starting from a racemic glufosinate mixture, meaning a composition would be obtained, which would consist substantially of L-glufosinate, there is still a flaw to the process to be noticed.

The full picture of the reaction reveals that L-glufosinate ammonium of formula L-(1) is not obtained at a stoichiometric rate, but that as a side product, an amine donor ammonium salt of the L-glufosinate as well.

For instance, if the amine donor is isopropylamine as shown in scheme 1a, then this results in a mixture of salts, namely the desired L-glufosinate-ammonium of formula L-(1) and the non-registred L-glufosinate-isopropylammonium of formula L-(2).

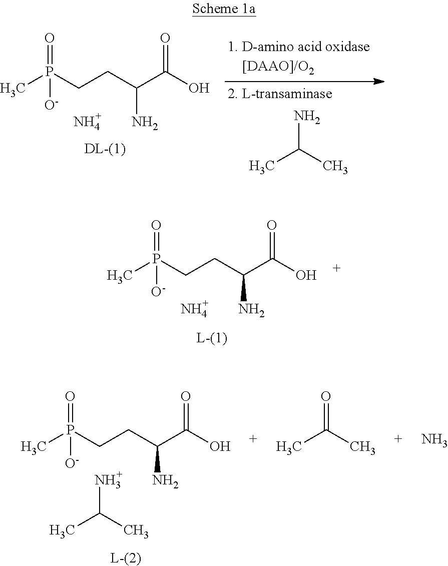

Hence, although the obtained L-glufosinate ammonium salt L-(1) can then be purified or substantially purified and used as a herbicide, the L-glufosinate-isopropylammonium salt of formula L-(2), would need to be further processed in order to benefit from it as well and obtain the desired L-glufosinate ammonium salt as well.

In general, the further processing of the L-glufosinate side products has the potential, depending on their nature, to become tedious, elaborate and time-consuming to an extent that it renders the process as whole unattractive.

As it is stated in WO 2017151573, the selection of an appropriate amine donor is important for an economical conversion of D-glufosinate to L-glufosinate. A variety of issues is listed, such as the availability and cost of the donor, the potential recovery of the donor and the separation of the side products, for example the keto side product, from the desired L-glufosinate.

It is recommended in WO 2017151573, that transaminase enyzmes accepting several different amine donors can be used, including low cost amine donors such as phenylethylamine, L-aspartate or racemic aspartate, L-glutamate or racemic glutamate, L-alanine or racemic alanine, sec-butylamine and isopropylamine. The latter is said to be optionally advantageous since removal of the co-product acetone from L-glufosinate can drive the reaction to completion. However, several, sometimes subsequent, problems are still to be faced:

For example, if the side product of the amination, e.g. the keto compound resulting from the donor molecule, cannot be removed continuously from the reaction mixture, firstly the amine donor has to be applied in huge excess in order to shift the equilibrium of the reaction to drive the desired reaction to completion, and secondly the later removal of amine donor excess from the desired L-glufosinate requires additional purification steps.

However, even if the keto compound resulting from the donor molecule, can be easily removed continuously from the reaction mixture, like it is the case for acetone resulting from isopropylamine, other subsequent problems are to be faced.

For instance, if an alkylamine is used as the donor, this alkylamine represents a stronger base than ammonia and therefore, as a non-desired side reaction, the displacement of ammonia from the starting ammonium salt is observed. This leads to the formation of alkylammonium salts of L-glufosinate which—due to missing approval of the regulatory authorities—cannot be used for agricultural purpose and for commercial compositions.

In view thereof, the preferred amino donor chosen in the prior art was glutamate, as the keto acid co-product that results from the transamination reaction, namely α-ketoglutarate, can be isolated and/or purified with well established and known methods and can be further used in a variety of applications, including in synthesizing pharmaceutical agents, food additives, and biomaterials. Further it is mentioned, that it can be chemically converted to either racemic glutamate or L-glutamate, optionally for reuse in the reaction and chemical reductive amination would involve the conversion of a keto group to an amine.

However, although mentioned, ispropylamine was not considered as a preferable amine donor. In addition, as indicated above, it is not evident to find easily a wild type transaminase enzyme that can be used right away. Very often such a wild-type transaminase that does not normally accept a desired amine donor and would need to be evolved in order to finally accept the desired substrate, which may present a further challenge.

In view of the above illustrated problems, the problem to be solved is both: First to effectively separate the unwanted amine donor salt of L-glufosinate from the mixture, and then additionally—even better—to convert it directly into the ammonium salt of L-glufosinate L-(1) and thereby to increase the yield of the desired ammonium salt.

The solution according to the present invention refers to the right selection of the amine donor on the one side, and to the economically and environmentally friendly step of removing the amine donor, and thereby obtaining higher stochiometric yield of the desired L-glufosinate ammonium salt.

Surprisingly, a new method was now found, which allowed an easy, simple, environmentally and cost efficient way, to obtain L-glufosinate, and especially the favorably desired ammonium salt of L-glufosinate from the D,L glufosinate racemate.

Thus, it was found that at first an amine donor needs to be selected from an aliphatic C₁-C₆-alkylamine. This would result in a L-glufosinate C₁-C₆-alkylammmonium salt of formula L-(2). The advantage of selecting such aliphatic C₁-C₆-alkylamine is, that due to their low boiling point, they can easily be removed by simple distillation.

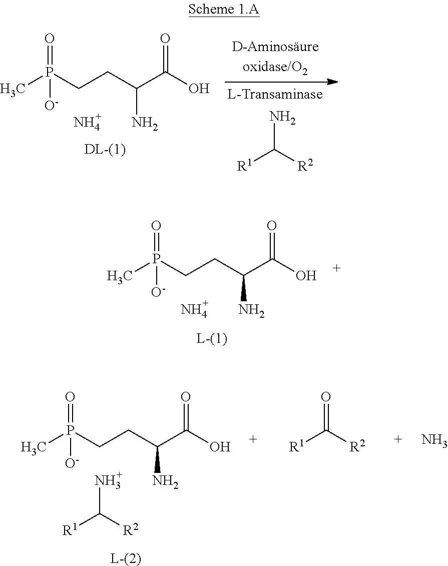

Thus, R¹ and R² in formula L-(2) would be selected independently from one another from the group consisting of hydrogen, methyl, ethyl, propyl, butyl and pentyl.

Preferably the aliphatic C₁-C₆-alkylamine is an aliphatic secondary C₁-C₆-alkylamine. More preferably the aliphatic secondary C₁-C₆-alkylamine would be selected from the group consisting of ethylamine, n-propylamine, isopropylamine, n-butylamine, sec-butylamine, amylamine, sec-pentylamine, n-hexylamine and sec-hexylamine.

The next challenge after the transamination would be to successfully separate the unwanted side product, the L-glufosinate C₁-C₆-alkylammmonium salt L-(2), from the desired L-glufosinate ammonium salt L-(1), and thereby preferably converting effectively the L-glufosinate C₁-C₆-alkylammmonium salt L-(2) into the desired L-glufosinate ammonium salt L-(1).

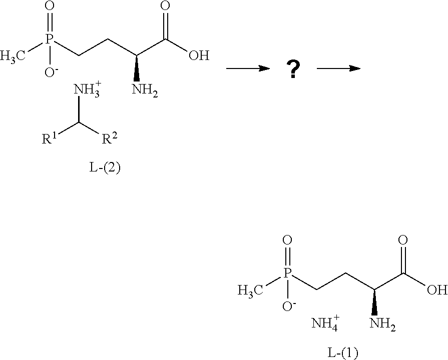

This problem can be solved by addition of Ca(OH)₂ in a first intermediate step, which converts the L-glufosinate alkylammmonium salt L-(2) into the calcium salt L-(3), and the hereby released low boiling C₁-C₆-alkylamine can be removed easily by distillation:

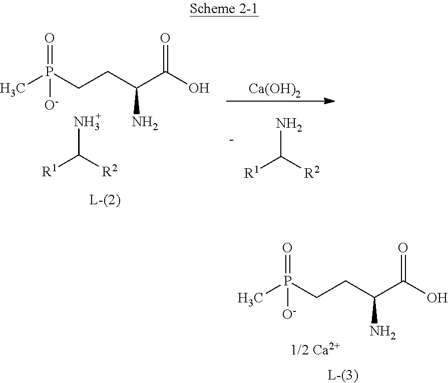

Being the weaker base, the alkylamine in the L-glufosinate C₁-C₆-alkylammmonium salt L-(2) is displaced from its salt by the stronger base Ca(OH)₂, and the calcium salt of L-glufosinate of formula L-(3) is formed. The released amine donor C₁-C₆-alkylamine having a low boiling point can then be removed by distillation.

In a second intermediate step, to the remaining aqueous solution of the calcium salt of L-glufosinate of formula L-(3), ammonium sulfate is to be added, which dissociates in the aqueous solution, and the sulfate ion forms the water-insoluble calcium sulfate (CaSO₄), commonly known as its hydrate gypsum or plaster, which precipitates, whereas the desired L-glufosinate ammonium salt L-(1) remains dissolved in the aqueous solution.

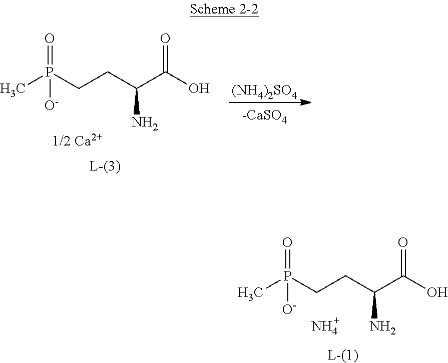

Gypsum is non-hazardous, non-toxic and inherently safe material, which can be simply removed by filtration. From the remaining aqueous solution, which now contains the desired L-glufosinate ammonium salt of formula L-(1), the pure salt can be obtained by removing the solvent and the ammonium salt L-(1) is obtained in an almost quantitative yield.

### EMBODIMENTS OF THE INVENTION

Individual and preferred embodiments of the methods for the conversion of D-glufosinate to L-glufosinate are given herein below.

The embodiments incorporate favorable and preferred means for converting a low cost feed-stock of a racemic mixture of D- and L-glufosinate into a herbicidal product, wherein the herbicidally active L-enantiomer L-glufosinate has been considerably enriched.

The method according to the present invention include means and methods for conversion as well as means for enriching the share of the desired L-enantiomer and its isolation.

The methods include several steps, which can occur consecutively either in one single container (one-pot-process) or consecutively in several separate containers (multi-pot-process).

### The DAAO Enzyme

The first step is the oxidative deamination of D-glufosinate (which can be present in a racemic mixture of D- and L-glufosinate) to PPO (2-oxo-4-(hydroxy(methyl)phosphinoyl)butyric acid).

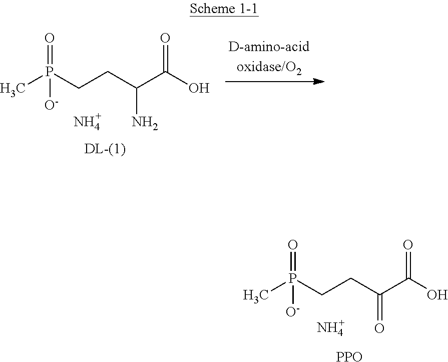

This step is preferably catalyzed by a D-amino acid oxidase (DAAO) enzyme.

However, the oxidative deamination of D-glufosinate to PPO, can be catalyzed by several classes of enzymes, but can also occur non-enzymatically. Possible enzymes include DAAO, DAAD (D-amino acid dehydrogenase (DAAD) enzyme), and D-amino acid dehydratase.

In one embodiment a DAAO enzyme is used to catalyze the conversion of D-glufosinate to PPO. Such a reaction has the following stoichiometry:

D-glufosinate+O₂+H₂O=>H₂O₂+NH₃+PPO.

Since the solubility of oxygen in aqueous reaction buffer is typically low compared to that of glufosinate, for an efficient process, oxygen must be introduced throughout the time period of the DAAO reaction.

Initially, D-glufosinate is present at greater than 30 g/L up to as much as 140 g/L. Oxygen is typically initially present at approximately 8 mg/L but is added throughout the reaction to allow for sufficient oxygen for the reaction to continue apace. Water is typically, but not obligately, present at greater than 500 g/L.

Several DAAO enzymes are known in the art and can be used in the methods described herein, as long as they are capable of accepting D-glufosinate as a substrate and provide an activity sufficient to level to drive the reaction. Such DAAO enzymes that can be used in the method include those from Rhodosporidium toruloides, Trigonopsis variabilis, Fusarium sp, Candida sp, Schizosasaccharomyces sp, Verticillium sp, Neolentinus lepideus, Trichoderma reesei, Trichosporon oleaginosus, and the like that have been modified to increase activity.

Particular starting enzymes have been described and identified in WO2017/151573, which is incorporated herein by reference in its entirety, here especially starting from page 7.

Additional DAAO enzymes can be identified in a variety of ways, including sequence similarity and functional screens. Here, if the DAAO enzyme is a mutant DAAO enzyme, it needs to be capable of accepting D-glufosinate as a substrate. Other DAAO enzymes can be similarly modified to accept D-glufosinate and have greater activity. In the same manner, known DAAO enzymes may be improved by mutagenesis, and/or novel DAAO enzymes could be identified.

In some embodiments, mutant enzymes can be made and tested in the methods described herein. Mutant DAAO enzymes (e.g., from Rhodotorula gracilis) can include one mutation, two mutations, three mutations, or more than three mutations (e.g., four mutations, five mutations, six mutations, seven mutations, eight mutations, nine mutations, or ten mutations or more) at positions in the mutant sequence as compared to the wild type sequence. Also here reference is made to the disclosure in WO2017/151573.

Other suitable D amino acid oxidases may be obtained from fungal sources.

As indicated, DAAO enzymes can be identified and tested for use in the methods of the invention. To determine if the enzyme will accept D-glufosinate as a substrate, an oxygen electrode assay (Hawkes, 2011), colorimetric assay (Berneman A, Alves-Ferreira M, Coatnoan N, Chamond N, Minoprio P (2010) Medium/High Throughput D-Amino Acid Oxidase Colorimetric Method for Determination of D-Amino Acids. Application for Amino Acid Racemases. J Microbial Biochem Technol 2: 139-146), and/or direct measurement (via high performance liquid chromatography (HPLC), liquid chromatography mass spectrometry (LC-MS), or similar) of product formation can be employed.

The reaction catalyzed by the DAAO enzyme requires oxygen. In some embodiments, oxygen, oxygen enriched air, an oxygen enriched gas stream, or air, is introduced to the reaction, either in the head space or by sparging gas through the reaction vessel, to enhance the rate of reaction.

When a DAAO enzyme catalyzes the conversion of D-glufosinate to PPO, hydrogen peroxide (H₂O₂) evolves. This may be damaging to enzymes and other components of the biotransformation (e.g., products and/or substrates). Therefore, in one embodiment, an enzyme, such as catalase, can be used in addition to the DAAO enzyme to catalyze the elimination of hydrogen peroxide. Catalase catalyzes the decomposition of hydrogen peroxide with the following stoichiometry: 2H₂O₂=>2H₂O+O₂.

In some embodiments, hydrogen peroxide can be eliminated using catalyzed and non-catalyzed decomposition reactions. For example, hydrogen peroxide can be eliminated by a non-catalyzed decomposition reaction using increased heat and/or pH. Hydrogen peroxide can also be eliminated by a catalyzed decomposition reaction using, for example, transition metals and other agents, such as potassium iodide. In addition to eliminating hydrogen peroxide, the use of catalase also produces oxygen (O₂). The production of oxygen by catalase can aid in facilitating the conversion of D-glufosinate to PPO using the DAAO enzyme, as DAAO requires oxygen to function.

Other enzymes can be used to catalyze the conversion of D-glufosinate to PPO. For example, a DAAD enzyme that accepts D-glufosinate as a substrate can be used with the following stoichiometry: D-glufosinate+H₂O+acceptor=>NH₃+reduced acceptor+PPO.

It is recognized that in methods where a DAAD is used, the DAAD catalyzed reaction can include redox cofactor recycling. This involves oxidizing the reduced acceptor so that it can accept more electrons from D-glufosinate.

The substantially complete (greater than 80%, greater than 85%, greater than 90%, or greater than 95% or from 80 to 100%, from 85 to 99%, from 90 to 99%, or from 95 to 99%) conversion of D-glufosinate to PPO can occur within 24 hours, within 18 hours, within 12 hours, or within 8 hours.

### The TA Enzyme

The second step is the specific amination of PPO with a transaminase (TA) enzyme to L-glufosinate ammonium salt of formula L-(1) and an alkylammoniumsalt of formula L-(2), using an amine group from one or more amine donors.

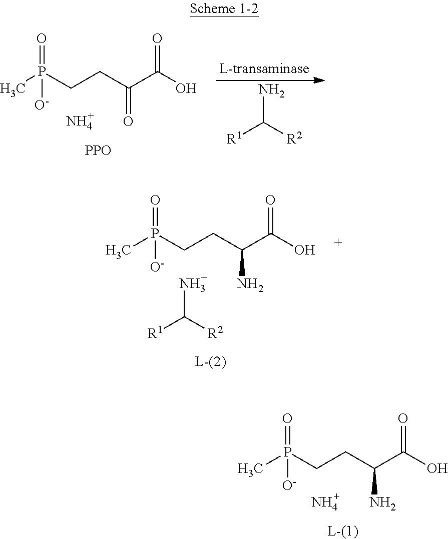

This second step involves the conversion of PPO to L-glufosinate using for instance a transaminase (TA) enzyme, an L-amino acid dehydrogenase (LAAD) enzyme or by chemical conversion. In one preferred embodiment, the method is a reaction catalyzed by a TA.

Transaminases (TAs) are in general important enzymes for the production of chiral amines for the pharmaceutical and fine chemical industries. Novel TAms for use in these industries have been discovered using a range of approaches, including activity-guided methods and homolo-gous sequence searches from cultured microorganisms to searches using key motifs and meta-genomic mining of environmental DNA libraries (Kelly et al. (2020) Appl Microbiol Biotech 104:4781-4794).

If the reaction is conducted as a two stage process where the D-glufosinate is substantially converted to PPO in the absence of amine donor and/or transaminase, starting amounts of PPO in the second stage typically range from 30 g/L to 140 g/L. If the reaction is conducted in a single stage process, the starting amounts of PPO are typically less than 1 g/L and the highest levels of PPO during the reaction are typically less than 25 g/L. The amine donor is initially present at between 1 and 6 molar excess over the starting amount of racemic glufosinate.

TAs useful in the methods described herein include the gabT transaminase from Escherichia coli (UniProt P22256), which has been shown to catalyze the desired reaction with PPO as a substrate (Bartsch et al. (1990) Appl Environ Microbiol. 56(1):7-12). Another enzyme has been evolved to catalyze the desired reaction at a higher rate using isopropylamine as an amine donor (Bhatia et al. (2004) Peptide Revolution: Genomics, Proteomics & Therapeutics, Proceedings of the Eighteenth American Peptide Symposium, Ed. Michael Chorev and Tomi K. Sawyer, Jul. 19-23, 2003, pp. 47-48). Additionally, TA enzymes from numerous microorganisms, such as Streptomyces hygroscopicus, Streptomyces viridochromogenes, Candida albicans, and others can be used in the practice of the methods described herein.

Where desired, the enzymes can be evolved as well by mutagenesis to increase their activities. Mutant TA enzymes can be selected for desired activity by the assays outlined in Schulz et al., Appl Environ Microbiol. (1990) Jan. 56(1):1-6, and/or by direct measurement of the products by HPLC, LC-MS, or similar products.

Additional TA enzymes for use in the methods can be identified by screening collections of TAs, such as those sold by Prozomix Limited (Northumberland, United Kingdom), SyncoZymes (Shanghai, China), Evocatal (Monheim am Rhein, Germany), Codexis (Redwood City, CA), or Abcam (Cambridge, United Kingdom) for the desired activity. Alternatively, sequence similarity can be used to identify novel TA enzymes.

Eventually, TA enzymes can also be identified from organisms capable of catalyzing the desired reaction and may even be further modified and optimized by enzyme engineering to allow economical production having better selectivity and higher output.

The latter is done e.g. in WO 2020/025577, incorporated herein by reference in its entirety, which describes proteins having improved omega-transaminase (ω-TA) activity, nucleic acid molecules encoding respective proteins having improved ω-TA activity and methods for stereo selective synthesis of chiral amines and amino acids or increasing of chiral amines isomers in enantiomer mixtures. Provided are especially ω-transaminases (ω-TAs) variants comprising modifications in their amino acid sequence having improved reaction kinetics, improved substrate acceptance and improved specific activity in comparison to respective wild-type ωTAs and therefore enabling the development of economically efficient production processes for aminated products. These variants can produce enantiomerically enriched, nearly pure or pure compounds of phospho-amino acids such as glufosinate.

Preferred ω-TA variants in WO2020025577 allow the production of compositions comprising an (S)-amine in enantiomeric excess, thus especially being suitable for obtaining (S)-glufosinate in enantiomeric excess, wherein the terminology of (S)-enantiomeric for glufosinate identifies the same desired L-glufosinate. Conveniently, respective ω-TA variants decrease at the same time the amount of (R)-enantiomers in the composition, thus less of the inactive D-enantiomer of glufosinate is obtained.

The substantially complete conversion of PPO to L-glufosinate may occur within 24 hours, within 18 hours, within 12 hours, or within 8 hours. Substantially complete, in this context, means that the conversion of PPO to L-glufosinate is greater than about 80%, greater than about 85%, greater than about 90%, greater than about 95%, greater than about 98%, or greater than about 99% (or from about 80 to about 100%, from about 85 to about 100%, from about 90 to about 100%, or from about 95 to about 99%).

### The Amine Donor

The amine donor molecule is an amine group comprising molecule which donates an amine group to the amine acceptor molecule, thereby an amine group of the amine donor molecule becoming a carbonyl group.

The amine donor molecule selected in the present invention is a C₁-C₆-alkyl-amine.

As mentioned further above, the amine donor is to be selected from an aliphatic C₁-C₆-alkylamine due to their low boiling point, as they can easily be removed by simple distillation. Preferably the amine donor is to be selected from a secondary aliphatic C₁-C₆-alkylamine. Hence, in one embodiment of the invention, the aliphatic secondary C₁-C₆-alkylamine is preferably selected from the group consisting of ethylamine, n-propylamine, isopropylamine, n-butylamine, sec-butylamine, amylamine, sec-pentylamine, n-hexylamine and sec-hexylamine. In a more preferred embodiment of the invention the aliphatic secondary C₁-C₆-alkylamine is either isopropylamine or sec-butylamine.

Especially preferred the aliphatic secondary C₁-C₆-alkylamine is isopropylamine, which will re-suit in the isopropylammonium salt of L-glufosinate of formula L-(2a).

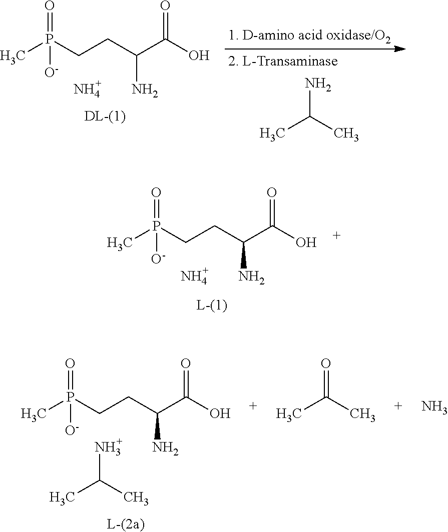

Kelly et al. (2017, Chem. Rev. 2018, 118, 1, 349-367), describes for instance that alanine has proven popular as an amine donor for TA-catalyzed reactions as to its widespread acceptance by enzymes and the various options to remove the pyruvate coproduct. However, it is pointed out, that the use of alanine results in an unfavorable reaction equilibrium, which is far on the side of the starting material. Later Kelly et al. ((2020) Appl Microbiol Biotech 104:4781-4794) describes the evolutionary diversity in the TAs with sequences well distributed throughout those of known S-selective TAs. One of those (called “pQR2189”) displayed the ability to accept the amino donor isopropylamine (“IPAm”).

The advantage of isopropylamine is not only it's acceptable chemical price and the ease with which by products can be removed, but also a significant advance in improvement of conversion rates.

Isopropylamine when used as an amino donor molecule in the methods according the invention, is converted by the action of an ω-TA into acetone. Acetone is a volatile compound leading to the advantage that it evaporates at relatively low temperatures. This allows removing the acetone produced by the ω-TA from the reaction mixture during the reaction taking place leading to the advantageous effect that the equilibrium of the reaction is shifted towards the desired amine produced by the method for production of an amine according to the invention. This allows obtaining the desired amine in high amounts as the reverse reaction catalyzed by ω-TA is reduced due to lack of one reaction partner.

Previously mentioned WO2020025577 refers as well to the alkylamines used as amino donor molecule such as 2-butylamine and isopropylamine.

Isopropylamine in its role as amine donor for TA can also be found in other publications such as in Park et al. (2013, Organic & Biomolecular Chemistry 11, 6929-6933), which discloses the behaviour of different transaminases in enantioselective synthesis of unnatural amino acids from keto acids by using isopropylamine and various other compounds as amine donors.

Preferably, the amine donor molecule for production of an amine is provided in an amount of between 10 g/l (gram per litre) to 250 g/l, more preferably between 15 g/l to 200 g/l, further more preferably between 17 g/l to 180 g/l.

### EXEMPLARY EMBODIMENTS

Herein below exemplary, but non-limiting, embodiments are described to further define the invention.

A method for obtaining L-glufosinate ammonium salt of formula L-(1) from a L-glufosinate C₁-C₆-alkylammonium salt of formula L-(2),

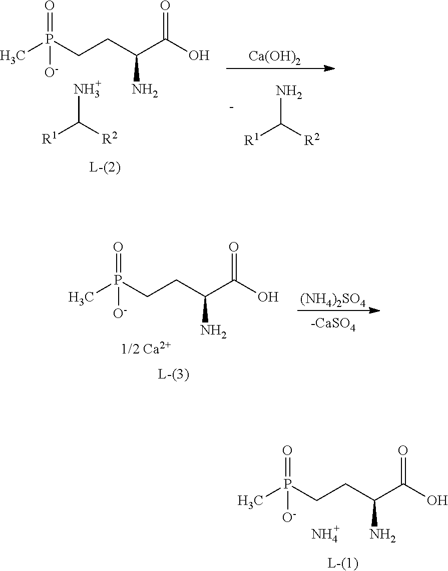

wherein

R¹ and R² in formula L-(2) are selected independently from one another from hydrogen, methyl, ethyl, propyl, butyl or pentyl; and

which method characterized by the following steps:

Step 1: adding Ca(OH)₂ to an aqueous solution of a L-glufosinate C₁-C₆-alkylammonium salt L-(2), whereby the L-glufosinate C₁-C₆-alkylammonium salt (L-2) reacts to the L-glufosinate calcium salt L-(3), and the thereby released low boiling C₁-C₆-alkylamine is removed by distillation Step 2: adding (NH₄)₂SO₄ to the remaining aqueous solution of the calcium salt of L-glufosinate L-(3), whereby the calcium ion of the L-glufosinate L-(3) calcium is replaced by the ammonium ion of the ammonium sulfate, and the precipitated calcium sulfate (gypsum) is removed by filtration.

Step 3: isolating the desired ammonium salt L-(1) by removal of water from the clear filtrate.

A method as described above, wherein the distillation in step 1 is performed at a temperature of more than 70 to 130° C., preferably of 75 to 120° C., and in particular of 80 to 110° C. In this connection, the distillation is preferably performed under atmospheric pressure (also referred to as normal pressure). The distillation may also be performed under reduced pressure, preferably from 100 mbar to below atmospheric pressure, more preferably from 100 mbar to less than 1000 mbar, and in particular from 100 mbar to 900 mbar. In this connection, the distillation can be performed at a temperature of 20 to 110° C. or of 30 to 120° C.

A method as described above, wherein step 2 further comprises the addition of an alcohol, preferably selected from the group consisting of methanol, ethanol, propanol, isopropanol, butanol, and mixtures thereof, and in particular methanol. In this connection, the alcohol is preferably added at a temperature of 25 to 75° C., more preferably of 30 to 70° C., even more preferably of 35 to 65° C., and in particular of 40 to 60° C.

A method as described above, wherein step 2 is conducted subsequent to step 1 without a filtration step.

A method as described above, wherein the L-glufosinate-alkylammonium salt of formula L-(2) is obtained by deracemization of glufosinate-ammonium of formula DL-(1) in a two-step process,

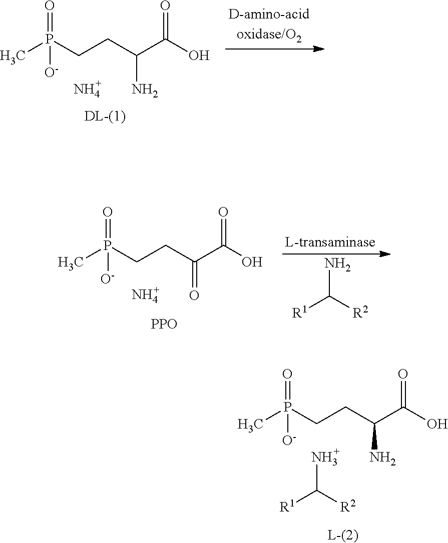

wherein in a first step the oxidative deamination of D-glufosinate to PPO (2-oxo-4-(hydroxy(methyl)phosphinoyl)butyric acid) is carried out with a D-amino acid oxidase (DAAO) enzyme, and

in a second step the PPO is aminated to a L-glufosinate C₁-C₆-alkylammonium salt of formula L-(2) by a transaminase (TA) enzyme using an amine group from one or more amine donors.

A method as described above, wherein the L-glufosinate C₁-C₆-alkylammonium salt of formula L-(2) is obtained in the second step by reaction with the transaminase (TA) enzyme in the presence of an amine donor, which is an aliphatic secondary C₁-C₆-alkylamine selected from the group consisting of ethylamine, n-propylamine, isopropylamine, n-butylamine, sec-butylamine, amylamine, sec-pentylamine, n-hexylamine and sec-hexylamine. Preferably, wherein the amine donor is a secondary aliphatic C₁-C₆-alkylamine selected from isopropylamine or sec-butylamine, more preferably isopropylamine.

A method as described herein above, wherein C₁-C₆-alkylammonium ion in the L-glufosinate C₁-C₆-alkylammonium salt of formula L-(2) is an ammonium salt of the aliphatic C₁-C₆-alkylamine as described herein above. Preferably, wherein C₁-C₆-alkylammonium ion in the L-glufosinate C₁-C₆-alkylammonium salt of formula L-(2) is isopropylamine ammonium or sec-butylamine ammonium, more preferably isopropylamine ammonium.

The method as described above, wherein the DAAO enzyme is selected from an enzyme from Rhodosporidium toruloides (UniProt P80324),Trigonopsis variabilis (UniProt Q99042), Neolentinus lepideus (KZT28066.1), Trichoderma reesei (XP_006968548.1), or Trichosporon oleaginosus (KLT40252.1).

Preferably, the DAAO enzyme is a mutant DAAO and more preferably the mutant DAAO is a mutant DAAO based on the sequence from Rhodosporidium toruloides.

The method as described above, wherein the TA enzyme is a ω-transaminase.

Preferably the TA enzyme is a S-selective ω-transaminase enzyme selected from Arthrobacter sp., Bacillus megaterium, Klebsiella pneumoniae JS2F (S), Bacillus thuringiensis JS64 (S), V. fluvialis JS17 (S), , Pseudomonas sp. KNK425 (S), Alcaligenes denitrificans Y2k-2 (S), Mesorhizobium sp. LUK (S),Bacillus megaterium SC6394 (S), Moraxella lacunata WZ34 (S), Janibacter terrae DSM13953 (S), Pseudomonas cichorii DSM 50259 (S), Pseudomonas fluorescens ATCC49838 (S), Pseudomonas fluorescens KNK08-18 (S) , Pseudomonas sp. ACC (S), Pseudomonas putida NBRC14164 (S), Bacillus halotolerans (S), Bacillus subtilis subsp. stercoris (S), Bacillus subtilis subsp. inaquosorum (S), Bacillus endophyticus (S), Rhizobium radiobacter (S), Chromobacterium violaceum DSM30191 (S), Pseudomonas aeruginosa (S) Ingram et al. (2007), Arthrobacter citreus (S), Caulobacter crescentus (S), Rhodobacter sphaeroides (S), Paracoccus denitrificans (S), Polaromonas sp. JS666 (S), Ochrobactrum anthropi (S), Acinetobacter baumannii (S) , Acetobacter pasteurianus (S), Burkholderia vietnamensis (S), Halomonas elongata (S), Burkholderia graminis (S), Thermomicrobium roseum (S), Sphaerobacter thermophilus (S), Geobacillus thermodenitrificans (S), Bacillus megaterium (S), Bacillus mycoides (S), Halomonas sp. CSM-2 (S), Rhodospirillaceae bacterium (S), Labrenzia sp. LAB (S), Afipia sp. P52-10 (S), Oceanibaculum indicum (S), Ilumatobacter coccineus (S), Variovorax sp. KK3 (S), Paraburkholderia caribensis (S), Hydrogenophaga palleronii (S), Solirubrobacter soli (S), Kineosporia sp. R_H_3 (S), Roseomonas deserti (S), Sinorhizobium meliloti (S), Bosea lupine (S), Bosea vaviloviae (S), Pseudacidovorax intermedius (S), Burkholderia sp. UYPR1.413 (S),), Escherichia coli K12 (S), Pseudomonas putida (S), Pseudomonas fluorescens (S), Pseudomonas chlororaphis (S), , Silicibacter pomeroyi (S), Rhodobacter sphaeroides KD131 (S), Ruegeria sp. TM1040 (S), Mesorhizobium loti MAFF30399 (S) or Bacillus anthracis (S).

More preferably the TA enzyme is a mutant ω-transaminase

Most preferably the TA is a mutant ω-transaminase based on the sequence from Arthrobacter sp. or from Bacillus megaterium.

Especially, the preferred selected ω-transaminase mutant is described in WO 2020/025577.

A method for obtaining L-glufosinate ammonium salt of formula L-(1) by deracemization of glufosinate-ammonium of formula DL-(1), wherein in a starting step the oxidative deamination of D-glufosinate to PPO (2-oxo-4-(hydroxy(methyl)phosphinoyl)butyric acid) is carried out with a D-amino acid oxidase (DAAO) enzyme, and in a following step the PPO is aminated to a L-glufosinate C₁-C₆-alkylammonium salt of formula L-(2) by a transaminase (TA) enzyme using an amine group from an amine donor selected from the group consisting of ethylamine, N-propylamine, isopropylamine, N-butylamine, sec-butylamine, amylamine, sec-pentylamine, n-hexylamine and sec-hexylamine,

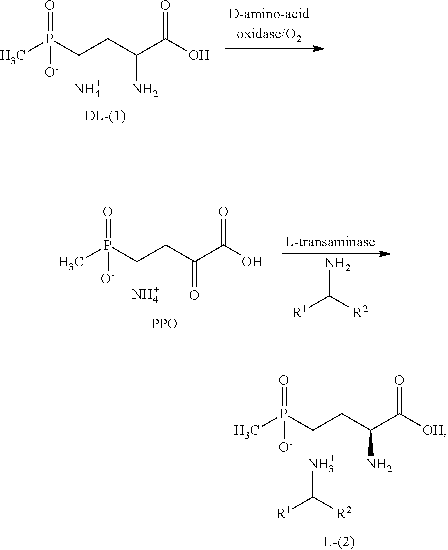

wherein R¹ and R² in formula L-(2) are selected from methyl, ethyl, propyl, butyl or pentyl, and in a further following step Ca(OH)₂ is added to the aqueous solution of L-glufosinate C₁-C₆-alkylammonium salt of formula L-(2), whereby it reacts to L-glufosinate calcium salt of formula L-(3), and the released low-boiling C₁-C₆-alkylamine is removed by distillation:

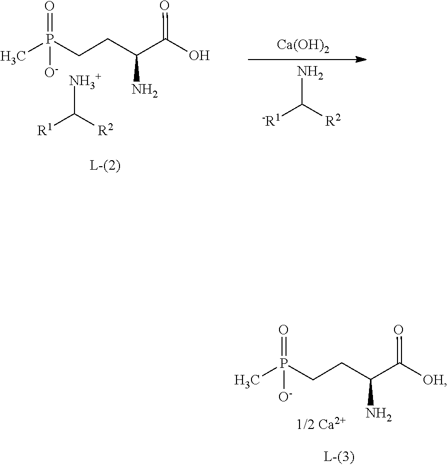

and in a further following step (NH₄)₂SO₄ is added to the remaining aqueous solution of the calcium salt of L-glufosinate of formula L-(3), whereby the calcium ion of the L-glufosinate calcium salt of formula L-(3) is replaced by the ammonium ion of the ammonium sulfate, and the precipitated calcium sulfate (gypsum) is removed by filtration:

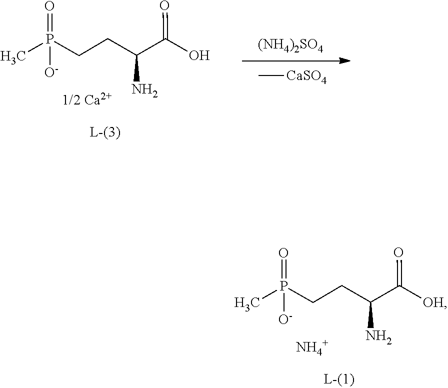

and wherein in a final step the desired L-glufosinate ammonium salt of formula L-(1) is obtained by removal of water from the clear filtrate.

The method as described herein above, wherein all method steps are performed consecutively as one-pot-process in a single container.

The method as described herein above, wherein the individual method steps are performed consecutively as multi-pot-process in separate containers.

A method as described above, wherein the distillation step is performed at a temperature of more than 70 to 130° C., preferably of 75 to 120° C., and in particular of 80 to 110° C. In this connection, the distillation is preferably performed under atmospheric pressure (also referred to as normal pressure). The distillation may also be performed under reduced pressure, preferably from 100 mbar to below atmospheric pressure, more preferably from 100 mbar to less than 1000 mbar, and in particular from 100 mbar to 900 mbar. In this connection, the distillation can be performed at a temperature of 20 to 110° C. or of 30 to 120° C.

A method as described above, wherein when (NH₄)₂SO₄ is added, the step further comprises the addition of an alcohol, preferably selected from the group consisting of methanol, ethanol, propanol, isopropanol, butanol, and mixtures thereof, and in particular methanol. In this connection, the alcohol is preferably added at a temperature of 25 to 75° C., more preferably of 30 to 70° C., even more preferably of 35 to 65° C., and in particular of 40 to 60° C.

A method as described above, wherein the (NH₄)₂SO₄ addition is conducted subsequent to the distillation step without a filtration step.

A method for obtaining an ammonium salt of an organic carboxylic acid RCOONH₄ of formula (6) having the organic moiety R being an (hetero)aromatic or (hetero)aliphatic, (hetero)cyclic or straight/branched open-chained, saturated or unsaturated moiety, optionally comprising one or more heteroatoms,

which method is characterized by
(A) a first step, wherein an intermediate calcium salt of the carboxylic acid of formula (5) is produced from a C₁-C₆-alkylammonium salt of the carboxylic acid of formula (4), wherein R¹ and R² in formula (4) are selected from independently from one another from hydrogen, methyl, ethyl, propyl, butyl or pentyl;

by adding Ca(OH)₂ and subsequently removing the displaced C₁-C₆-alkylamine by distillation, and wherein
(B) in a second step, the ammonium salt of the carboxylic acid RCOONH₄ of formula (6) is obtained by displacing the calcium ion of the carboxylic acid calcium salt of formula (5) by adding ammonium sulfate and subsequently removing the precipitated gypsum CaSO₄ by filtration:

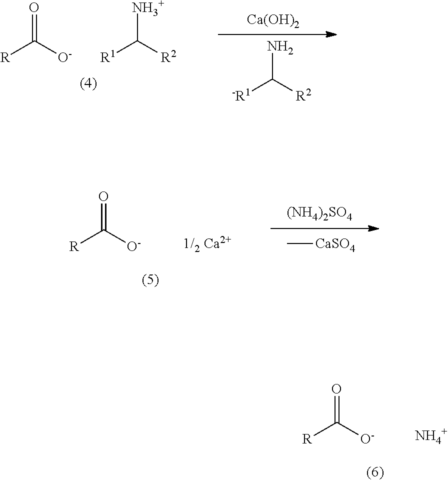

The method as described herein above, wherein the intermediate calcium salt of the carboxylic acid of formula (5) is produced from a secondary C₁-C₆-alkylammonium salt of the carboxylic acid of formula (4), wherein R₁ and R₂ in formula (4) are selected independently from one another from methyl, ethyl, propyl, butyl or pentyl.

The method as described herein above, wherein the distillation in step (A) is performed at a temperature of more than 70 to 130° C., preferably of 75 to 120° C., and in particular of 80 to 110° C. In this connection, the distillation is preferably performed under atmospheric pressure (also referred to as normal pressure). The distillation may also be performed under reduced pressure, preferably from 100 mbar to below atmospheric pressure, more preferably from 100 mbar to less than 1000 mbar, and in particular from 100 mbar to 900 mbar. In this connection, the distillation can be performed at a temperature of 20 to 110° C. or of 30 to 120° C.

The method as described herein above, wherein step (B) further comprises the addition of an alcohol, preferably selected from the group consisting of methanol, ethanol, propanol, isopropanol, butanol, and mixtures thereof, and in particular methanol. In this connection, the alcohol is preferably added at a temperature of 25 to 75° C., more preferably of 30 to 70° C., even more preferably of 35 to 65° C., and in particular of 40 to 60° C.

The method as described herein above, wherein step (B) is conducted subsequent to step (A) without a filtration step.

### METHODS OF PREPARATION

As noted above, the method of preparation of L-glufosinate according to the present invention involves a several step process, which process is characterized by the use of a calcium comprising strong base such Ca(OH)₂ for replacing the weaker (C₁-C₆)-alkylammonium ion in the L-glufosinate salt by calcium in a first step, and subsequently removing the released (C₁-C₆)-alkylamin having a low boiling point by distillation. And then, in a second step, adding to the remaining aqueous solution of the calcium salt of the L-glufosinate L-(3), from which the isopropylamine had been removed, ammonium sulfate.

Ammonium sulfate dissociated in the aqueous solution, and the sulfate ion forms the water-insoluble calcium sulfate (CaSO₄), commonly known as gypsum, which precipitates, and the desired ammonium salt L-(1) is obtained in an almost quantitative yield:

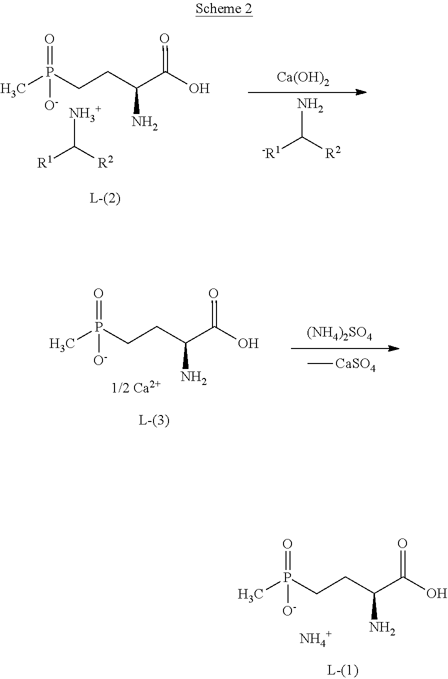

The precipitated gypsum (or plaster) can be easily removed from the solution by filtration, and from the remaining aqueous solution, which contains the ammonium salt L-(1), the pure salt can be obtained by removing the solvent.

In a preferred embodiment of the present invention, as illustrated in scheme 2a, the preferred amine donor is isopropylamin, which has a boiling point of 31.4° C., and can therefore be easily removed from the aqueous solution.

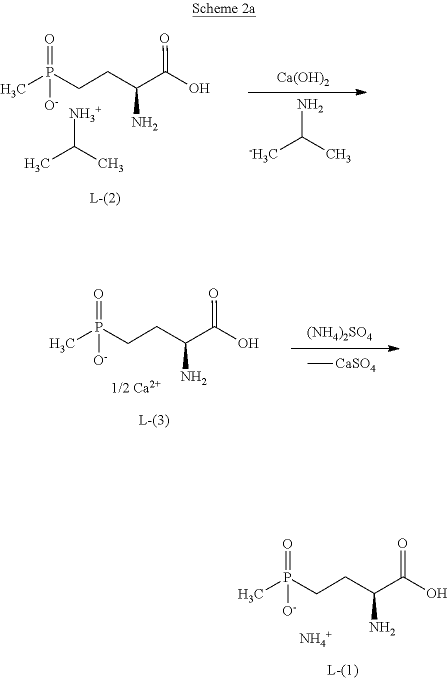

This new and inventive process step is embedded in the deracemization process of D,L glufosinate, which starts with the aforementioned oxidative deamination of D-glufosinate to PPO. Although after a subsequent transamination reaction step L-glufosinate can be obtained directly from PPO, a considerable amount of side product, namely L-glufosinate isopropylammonium of formula L-(2a), is obtained as well, which shall then be further processed according to the present invention as outlined herein above.

Although, as described herein above, the inventive method is used and applied for the preparation of L-glufosinate, the conversion is universally applicable and is not limited to glufosinate or phosphorous compounds in specifically, but—as shown in scheme X below—can also be easily applied for obtaining any ammonium salt of a carboxylic acid of formula (6), wherein R is an organic (hetero)aromatic or (hetero)aliphatic, a (hetero)cyclic or straight/branched open-chained, any saturated or unsaturated moiety optionally comprising one or more heteroatoms. Here, likewise, an intermediate Ca salt (5) is produced from a C₁-C₆-alkylammonium salt of the carboxylic acid of formula (4), wherein R¹ and R² in formula (4) are selected independently from one another from hydrogen, methyl, ethyl, propyl, butyl or pentyl; by adding Ca(OH)₂ and subsequently removing the displaced C₁-C₆-alkylamine by distillation, and wherein in a second step, the desired (unsubstituted) ammonium salt (6) of the carboxylic acid can be obtained as well by adding first ammonium sulfate, and subsequently removing the precipitated gypsum by filtration:

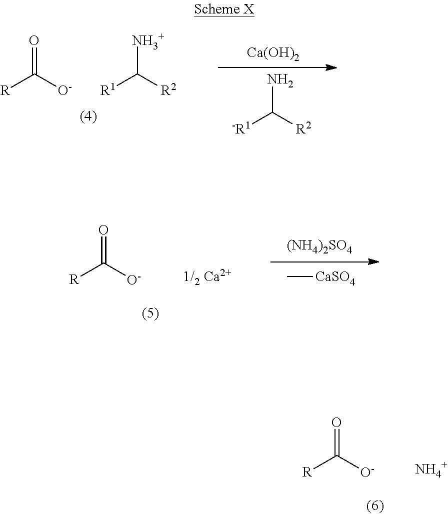

R in scheme X can be any organic moiety and R¹ and R² in formula (4) are selected independently from one another from hydrogen, methyl, ethyl, propyl, butyl or pentyl.

For individual illustration and representation purpose, herein below a preparation example is given in scheme Xa, wherein the salt is the isopropylammonium salt of 2-phenylpropanoic acid (7), meaning the anion is a non-phosphorous carboxylate, which forms with the intermediate Ca salt (8) after addition of Ca(OH)₂ and removal of isopropylamine, and which converts to the ammonium salt of 2-phenylpropionic acid (9) after addition of ammonium sulfate and precipitation of gypsum:

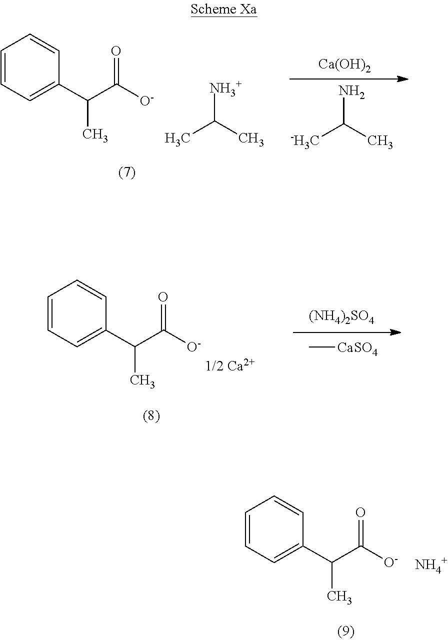

The process may be done in “one-pot” or in multiple containers, i.e. in separate transformations. If the reaction occurs in a single container or vessel, the TA enzyme can be added with the DAAO enzyme or added at a later time, e.g., after the DAAO enzyme has been allowed to catalyze some or substantially all of the oxidative deamination.

The enzymes can be added to the reaction by a number of methods.

One approach is to express the enzyme(s) in microorganism(s) such as E. coli, S. cerevisiae, P. pastoris, and others, and to add the whole cells to the reactions as whole cell biocatalysts. Another approach is to express the enzyme(s), lyse the microorganisms, and add the cell lysate. Yet another approach is to purify, or partially purify, the enzyme(s) from a lysate and add pure or partially pure enzyme(s) to the reaction. A further approach, which can be combined with the above approaches, is to immobilize enzyme(s) to a support (exemplary strategies are outlined in Datta et al. (2013) 3 Biotech. Feb; 3(1): 1-9). If multiple enzymes are required for a reaction, the enzymes can be expressed in one or several microorganisms, including expressing all enzymes within a single microorganism.

If multiple enzymes are required for a reaction, the enzymes can be expressed in one or several microorganisms, including expressing all enzymes within a single microorganism.

A further approach, which can be combined with the above approaches, is to immobilize enzyme(s) to a support (exemplary strategies are outlined in Datta et al. (2013) 3 Biotech. Feb; 3(1): 1-9). As outlined in Datta et al., and not intending to be limiting, enzymes, either singly or in combination, can, for example, be adsorbed to, or covalently or non-covalently attached to, or entrapped within, natural or synthetic polymers or inorganic supports, including aggregates of the enzyme(s) themselves. Once immobilized, the enzyme(s) and support can be dispersed into bulk solution or packed into beds, columns, or any number of similar approaches to interacting reaction solution with the enzymes. Since aeration is important for the DAAO reaction envisioned here, bubble columns or similar may be used for enzyme immobilization. As examples, reaction mixture can be flowed through a column of immobilized enzymes (flow reaction), added to a fixed bed or column of immobilized enzymes, allowed to react, and either removed from the bottom or top of the reaction vessel (plug flow), or added to dispersed immobilized enzymes and allowed to react then the immobilized enzymes removed by filtration, centrifugation, or similar (batch). Thus, any method for immobilization of the enzymes may be employed in the methods of the invention.

The DAAO, TA, and/or other reactions can occur in a buffer.

Exemplary buffers commonly used in biotransformation reactions include Tris, phosphate, or any of Good's buffers, such as 2-(N-morpholino)ethanesulfonic acid (MES); N-(2-Acetamido)iminodiacetic acid (ADA); piperazine-N,N′-bis(2-ethanesulfonic acid) (PIPES); N-(2-Acetamido)-2-aminoethanesulfonic acid (ACES); β-Hydroxy-4-morpholinepropanesulfonic acid (MOPSO); cholamine chloride; 3-(N-morpholino)propanesulfonic acid (MOPS); N,N-Bis(2-hydroxyethyl)-2-aminoethanesulfonic acid (BES); 2[[1,3-dihydroxy-2-(hydroxymethyl)propan-2-yl]amino]ethanesulfonic acid (TES); 4-(2-hydroxyethyl)-1-piperazineethanesulfonic acid (HEPES); 3-(Bis(2-hydroxyethyl)amino)-2-hydroxypropane-1-sulfonic acid (DIPSO); acetam-idoglycine, 3-(N-Tris(hydroxymethyl)methylamino(-2-hydroxypropanesulfonic acid (TAPSO); Piperazine-N,N′-bis(2-hydroxypropanesulfonic acid) (POPSO); 4-(2-Hydroxyethyl)piperazine-1-(2-hydroxypropanesulfonic acid) (HEPPSO); 3-[4-(2-Hydroxyethyl)-1-piperazinyl]propanesulfonic acid (HEPPS); tricine; glycinamide; bicine; or 3-[[1,3-dihydroxy-2-(hydroxymethyl)propan-2-yl]amino]propane-1-sulfonic acid (TAPS). Additional exemplary buffer recipes can be found in Whittall, J. and Sutton, P. W. (eds) (2012) Front Matter, in Practical Methods for Biocatalysis and Biotransformations 2, John Wiley & Sons, Ltd, Chichester, UK. Ammonium can also act as a buffer.

The DAAO, TA, and/or other reactions can occur with no or low levels (less than 1 mM) of buffer added (other than ammonium present due to the addition of racemic glufosinate ammonium). In particular, immobilized DAAO and TA may be stable and active in the presence of less than 1 mM phosphate buffer and with no other buffer except the ammonium present due to the addition of racemic glufosinate ammonium.

In some embodiments, the reaction occurs within a defined pH range, which can be between pH 4 to pH 10 (e.g., between pH 6 and pH 9, such as approximately pH 7.5 to pH 8).

In some embodiments, the reaction occurs at a defined temperature. The temperature can be kept at a point between room temperature and the boiling point of the solvent, most typically between room temperature and 50° C.

As indicated, the methods described herein provide a composition of substantially pure L-glufosinate (rather than a racemic mixture of L-glufosinate and D-glufosinate). Substantially pure L-glufosinate means that greater than about 80%, greater than about 85%, greater than about 90%, greater than about 95%, greater than about 96%, greater than about 97%, greater than about 98%, or greater than about 99% of the D-glufosinate has been converted to L-glufosinate resulting in a composition having greater than about 80%, greater than about 85%, greater than about 90%, greater than about 95%, greater than about 96%, greater than about 97%, greater than about 98%, or greater than about 99% L-glufosinate compared to the sum of the D-glufosinate and the L-glufosinate present in the composition. In this connection, it is to be understood that the term “greater than” refers to a range of up to 100%.

Optionally, the L-glufosinate can be partially or completely purified from the biotransformation reaction mixture. A number of alternative procedures can be employed, including, but not limited to: a) crystallization from the biotransformation mixture using suitable solvents such as ether or water at temperatures varying between −20° C. to the boiling point of the solution, b) concentration in vacuo of the biotransformation mixture followed by crystallization of the thus produced concentrate using suitable solvents such as ether or water at temperatures varying between −20° C. to the boiling point of the solution, or c) concentration in vacuo of the biotransformation mixture followed by fractional purification over an immobilized phase such as silica gel or Octadecyl silane ligands of the thus produced concentrate as a mobile phase in a solvent such as DCM, methanol (MeOH), EtOAc, ammonia (NH₃), or mixtures thereof, such as DCM/MeOH or EtOAC/MeOH/NH₃. Alternatively, the biotransformation mixture can be used directly (and/or with the addition of various adjuvants) for the prevention or control of weeds. Enzymes can be removed by simple filtration if supported, or if free in solution by the use of ultrafiltration, the use of absorbants like celite or carbon, or denaturation via various techniques known to those skilled in the art. The L-glufosinate can be isolated from the reaction mixture by ion-exchange chromatography or other solid phases known to be effective in retaining amino acids, or by crystallization as a cationic or anionic salt by adding a suitable organic or inorganic counter-ion known to form water insoluble salts of glufosinate. Such salts could be transformed into forms of glufosinate suitable for formulation by standard methods known to those skilled in the art. Alternatively, the L-glufosinate can be isolated as a Zwitter ion, cationic or anionic salt via crystallization by the addition of water miscible solvents, such as lower alcohols, ketones, tetrahydrofuran, acetonitrile. Alternatively, the glufosinate can be isolated by removal of water and dissolution in an organic solvent, such as methanol, desalting, then crystallization as the Zwitterion or conversion and subsequent crystallization as an acceptable salt form, such as HCI salt or ammonium salt. Alternatively, some or all of the components other than L-glufosinate can be removed from the biotransformation mixture, the mixture optionally concentrated, and then the mixture can be used directly (and/or with the addition of various adjuvants) for the prevention or control of weeds. Alternatively, the biotransformation mixture can be used directly (and/or with the addition of various adjuvants) for the prevention or control of weeds. Optionally, components, such as the amine donor, that remain unreacted can be partially or completely isolated and used in subsequent reactions. Optionally, unreacted PPO can be partially or completely isolated, chemically converted to racemic glufosinate, and used in subsequent reactions.

In one embodiment, the L-glufosinate is not isolated from the biotransformation mixture and a composition comprising D-glufosinate, PPO, and L-glufosinate is obtained. This composition may be used directly as a herbicidal composition.

It is understood that the terminology used herein is for the purpose of describing particular embodiments only, and the terminology is intended to be illustrating but not limiting. Unless defined otherwise, all technical and scientific terms used herein have the same meaning as commonly understood by one of ordinary skill in the art to which this invention belongs. Each of the individual embodiments described and illustrated herein has discrete components and features which may be readily separated from or combined with the features of any of the other several embodiments without departing from the scope or spirit of the invention. Any recited method may be carried out in the order of events recited or in any other order that is logically possible. Although any methods and materials similar or equivalent to those described herein may also be used in the practice or testing of the invention, representative illustrative methods and materials are now described.

The following examples are offered by way of illustration and not by way of limitation.

### EXAMPLES

### Figures

The effectivity of the preparation process according to the present invention for obtaining an ammonium salt of the desired enantiomer of a carboxylic acid is proven in the following figures as shown herein:

FIG. 1: ¹H-NMR spectrum of L-isopropyl-ammonium glufosinate L-(2):

FIG. 2: ¹H-NMR spectrum of L-ammonium glufosinate L-(1):

FIG. 3: ¹H-NMR spectrum of isopropyl-ammonium 2-phenylpropionate (7):

FIG. 4: ¹H-NMR spectrum of ammonium-2-phenylpropionate (9):

### Example 1

Conversion of L-isopropylammonium glufosinate L-(2) into L-ammonium glufosinate L-(1):

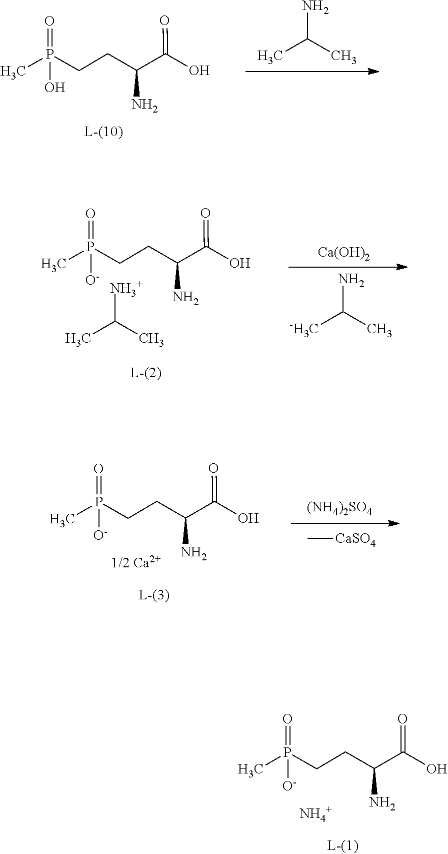

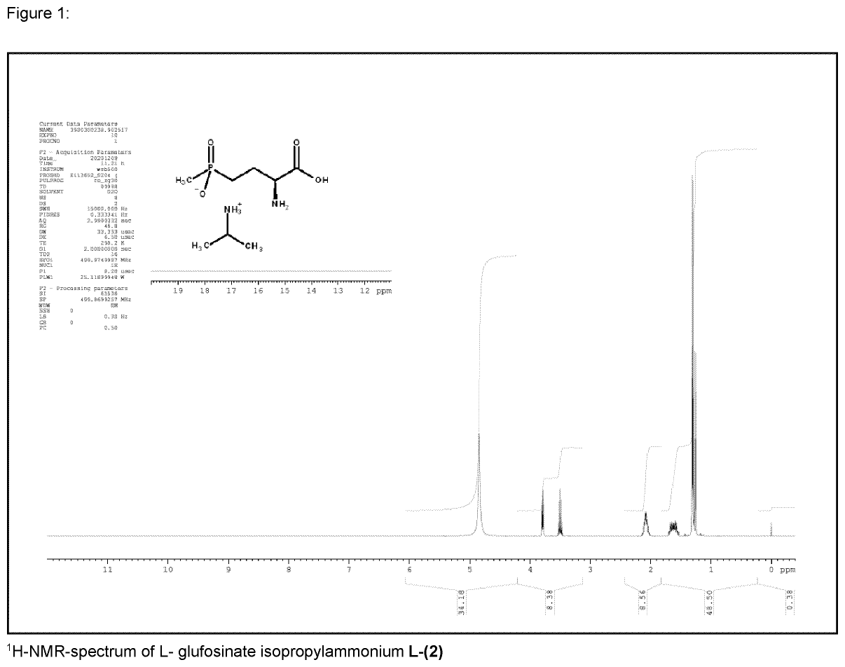

5.5 g (30 mmol) L-phosphonothricin L-(10) (Zeiss, H. -J.; J. Org. Chem. 1991, 56, 1783) were dissolved at room temperature in 80 ml of water and 1.9 g (33 mmol) isopropylamine. The solution was stirred for 1 hour at room temperature, a 0.5 ml sample of the clear solution was concentrated in vacuum and subjected to H-NMR analysis. The ¹H-NMR spectrum analysis of the obtained solid residue confirmed that isopropylammonium salt L-(2) had been obtained (see FIG. 1).

To the remaining solution 75 ml of water and 1.48 g (20 mmol) of calcium hydroxide was added. A slightly turbid solution of the calcium salt L-(3) was obtained. Under normal pressure, 100 ml water were removed from the solution by distillation. In the beginning, the boiling point was at 88-95° C., later. towards the end of the distillation, at 100° C. It could be shown by titration of the distillate with 1 N—H₂SO₄ that 1.85 g (which corresponds to 98%) of the initially obtained isopropylamine had been removed.

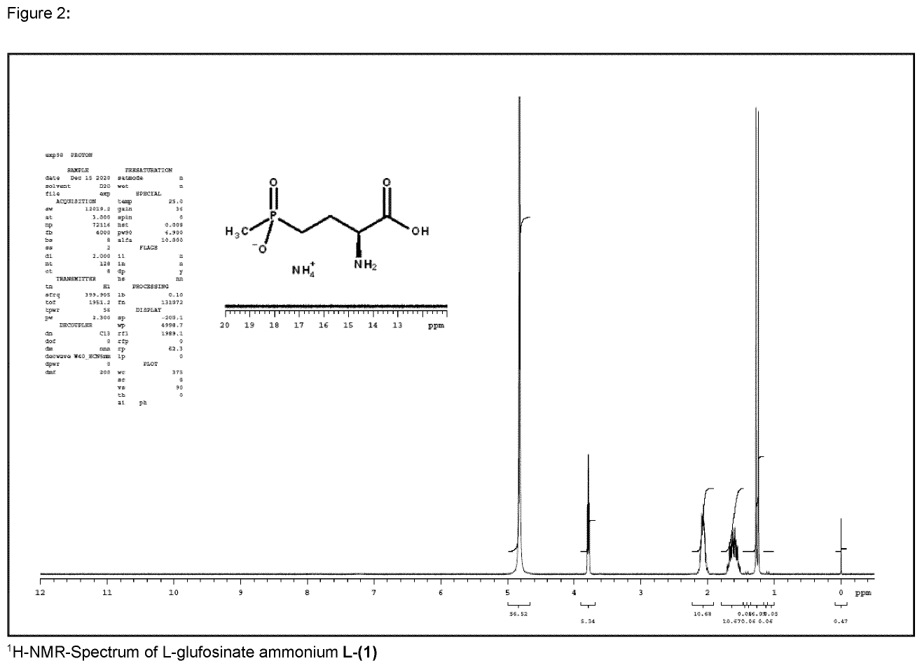

The remaining solution of Ca salt L-(3) was cooled to 50° C. and further diluted with 50 ml of methanol. 2.64 g (20 mmol) (NH₄)₂SO₄ was added to the solution, which was stirred overnight at room temperature, and the next day, the precipitated white solid gypsum was removed by filtration. The obtained clear filtrate was concentrated to dryness, and the white solid residue (¹H-NMR see FIG. 2) yielded in 6.2 g (96%) ammonium salt L-(1) (monohydrate) with a melting point of m.p.: 205° C. (decomposition). Its enantiomeric purity as levorotatory compound was proven via HPLC analysis as well as by its optical rotation ((−) direction).

### Example 2

Conversion of L-isopropylammonium 2-phenylpropionate (7) into ammonium 2-phenylpropionate (9):

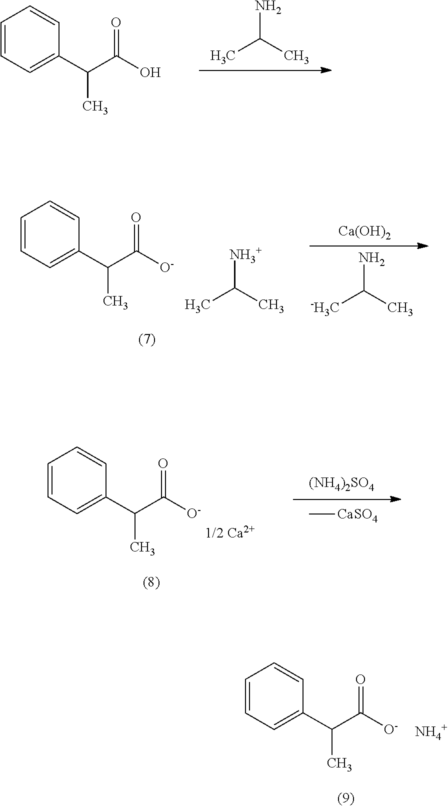

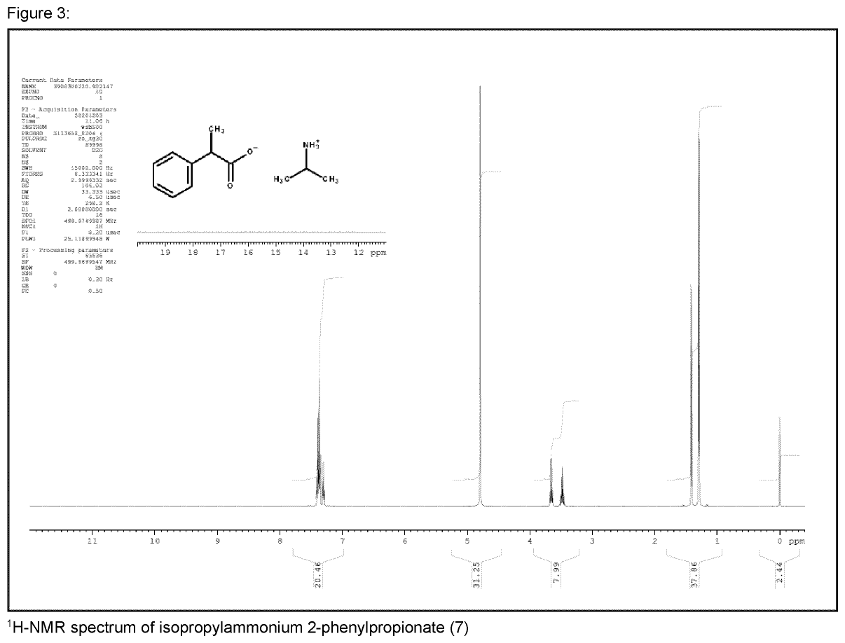

4.5 g (30 mmol) 2-phenylpropionic acid and 1.9 g (33 mmol) isopropylamine were dissolved in 80 ml of water at room temperature and the solution was stirred for 1 hour at room temperature. A 0.5 ml sample of the obtained clear solution was taken, concentrated in vacuum and subjected to H-NMR analysis. The ¹H-NMR spectrum analysis of the solid residue confirmed that the isopropylammonium salt (7) had been obtained (see FIG. 3).

The solution was diluted with 75 mL of water and 1.20 g (16 mmol) of calcium hydroxide was added. From this suspension of the calcium salt (8) 60 ml of water was removed by distillation at normal pressure. In the beginning the boiling point was 88-95° C., then, towards the end of distillation, at 100° C. The titration of the distillate with 1 N H₂SO₄ showed that 1.80 g (96%) isopropylamine had been removed with the water.

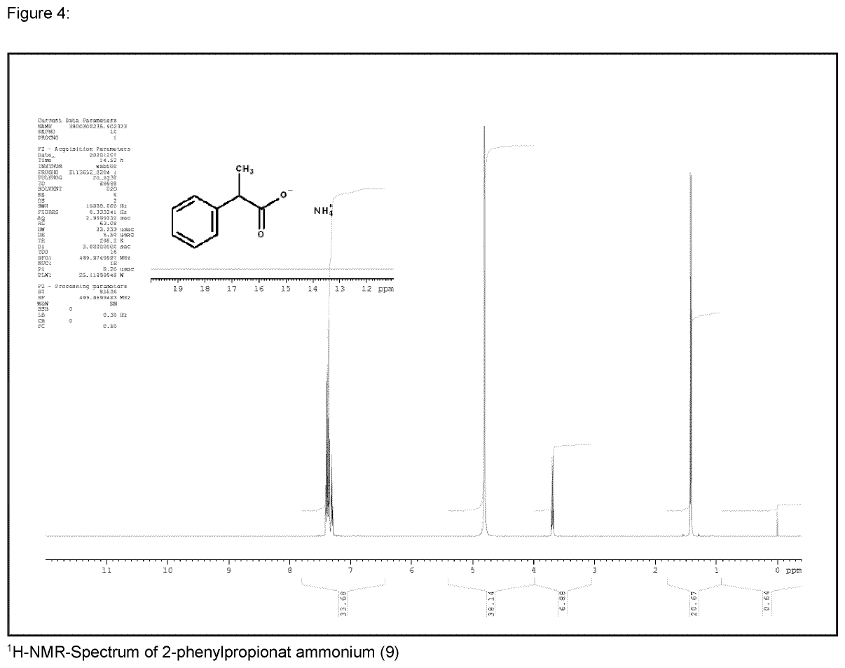

The remaining suspension of the Ca salt (8) was cooled to 50° C. and diluted with 50 ml methanol. 2.10 g (16 mmol) (NH₄)₂SO₄ was added to the suspension, which was then stirred overnight at room temperature. The next day the precipitated white solid gypsum was removed by filtration. The obtained clear filtrate was washed with 50 ml of methyl-t-butyl ether and the aqueous phase was further concentrated. The obtained colorless crystalline residue showed to be 4.2 g (84%) of the expected ammonium salt (9), which was confirmed by ¹H-NMR (see FIG. 4).

1. A method for obtaining L-glufosinate ammonium salt of formula L-(1) from a L-glufosinate C₁—C₆-alkylammonium salt of formula L-(2), 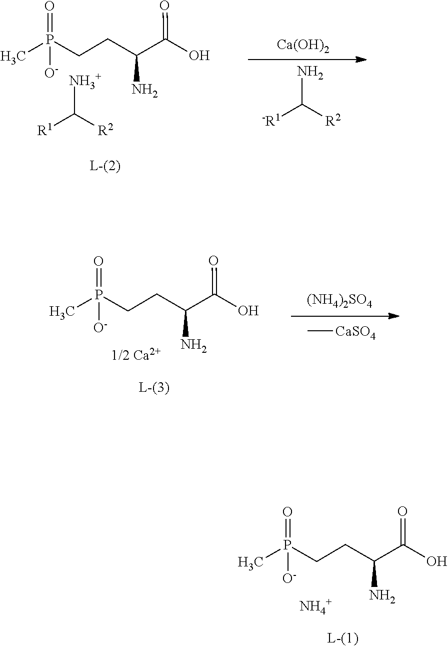
wherein R¹ and R² in formula L-(2) are selected independently from one another from hydrogen, methyl, ethyl, propyl, butyl, or pentyl; and
which method comprises:
Step 1: adding Ca(OH)₂ to an aqueous solution of a L-glufosinate C₁-C₆-alkylammonium salt L-(2), whereby the L-glufosinate C₁-C₆-alkylammonium salt (L-2) reacts to the L-glufosinate calcium salt L-(3), and the thereby released low boiling C₁-C₆-alkylamine is removed by distillation;
Step 2: adding (NH₄)₂SO₄ to the remaining aqueous solution of the calcium salt of L-glufosinate L-(3), whereby the calcium ion of the L-glufosinate L-(3) calcium is replaced by the ammonium ion of the ammonium sulfate, and the precipitated calcium sulfate (gypsum) is removed by filtration.
Step 3: isolating the desired ammonium salt L-(1) by removal of water from the clear filtrate.

2. The method according to claim 1, wherein the L-glufosinate-alkylammonium salt of formula L-(2) is obtained by deracemization of glufosinate-ammonium of formula DL-(1) in a two-step process, 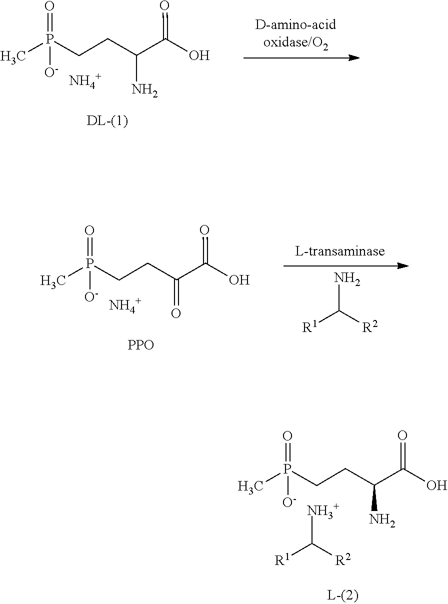
wherein in a first step an oxidative deamination of D-glufosinate to PPO (2-oxo-4-(hydroxy(methyl)phosphinoyl)butyric acid) is carried out with a D-amino acid oxidase (DAAO) enzyme, and
in a second step the PPO is aminated to a L-glufosinate C₁-C₆-alkylammonium salt of formula L-(2) by a transaminase (TA) enzyme using an amine group from one or more amine donors.

3. The method according to claim 2, wherein the L-glufosinate C₁-C₆-alkylammonium salt of formula L-(2) is obtained in the second step by reaction with the transaminase (TA) enzyme in the presence of an amine donor, which is an aliphatic secondary C₁-C₆-alkylamine selected from the group consisting of ethylamine, n-propylamine, isopropylamine, n-butylamine, sec-butylamine, amylamine, sec-pentylamine, n-hexylamine, and sec-hexylamine.

4. The method according to claim 2, wherein the amine donor is a secondary aliphatic C₁-C₆-alkylamine selected from isopropylamine or sec-butylamine.

5. The method according to claim 1, wherein the C₁-C₆-alkylammonium ion in the L-glufosinate C₁-C₆-alkylammonium salt of formula L-(2) is isopropylamine ammonium or sec-butylamine ammonium.

6. The method according to claim 2, wherein the DAAO enzyme is selected from an enzyme from Rhodosporidium toruloides (UniProt P80324), Trigonopsis variabilis (UniProt Q99042), Neolentinus lepideus (KZT28066.1), Trichoderma reesei (XP_006968548.1), or Trichosporon oleaginosus (KLT40252.1).

7. The method according to claim 2, wherein the DAAO enzyme is a mutant DAAO.

8. The method according to claim 6, wherein the mutant DAAO is a mutant DAAO based on the sequence from Rhodosporidium toruloides.

9. The method according to claim 2, wherein the TA enzyme is a S-selective ω-transaminase enzyme selected from the group consisting of Arthrobacter sp., Bacillus megaterium, Klebsiella pneumoniae JS2F (S), Bacillus thuringiensis JS64 (S), V. fluvialis JS17 (S), Pseudomonas sp. KNK425 (S), Alcaligenes denitrificans Y2k-2 (S), Mesorhizobium sp. LUK (S), Bacillus megaterium SC6394 (S), Moraxella lacunata WZ34 (S), Janibacter terrae DSM13953 (S), Pseudomonas cichorii DSM 50259 (S), Pseudomonas fluorescens ATCC49838 (S), Pseudomonas fluorescens KNK08-18 (S) , Pseudomonas sp. ACC (S), Pseudomonas putida NBRC14164 (S), Bacillus halotolerans (S), Bacillus subtilis subsp. stercoris (S), Bacillus subtilis subsp. inaquosorum (S), Bacillus endophyticus (S), Rhizobium radiobacter (S), Chromobacterium violaceum DSM30191 (S), Pseudomonas aeruginosa (S) Ingram et al. (2007), Arthrobacter citreus (S), Caulobacter crescentus (S), Rhodobacter sphaeroides (S), Paracoccus denitrificans (S), Polaromonas sp. JS666 (S), Ochrobactrum anthropi (S), Acinetobacter baumannii (S), Acetobacter pasteurianus (S), Burkholderia vietnamensis (S), Halomonas elongata (S), Burkholderia graminis (S), Thermomicrobium roseum (S), Sphaerobacter thermophilus (S), Geobacillus thermodenitrificans (S), Bacillus megaterium (S), Bacillus mycoides (S), Halomonas sp. CSM-2 (S), , Rhodospirillaceae bacterium (S), Labrenzia sp. LAB (S), Afipia sp. P52-10 (S), Oceanibaculum indicum (S), Ilumatobacter coccineus (S), Variovorax sp. KK3 (S), Paraburkholderia caribensis (S), Hydrogenophaga palleronii (S), Solirubrobacter soli (S), Kineosporia sp. R_H_3 (S), Roseomonas deserti (S), Sinorhizobium meliloti (S), Bosea lupine (S), Bosea vaviloviae (S), Pseudacidovorax intermedius (S), Burkholderia sp. UYPR1.413 (S), ), Escherichia coli K12 (S), Pseudomonas putida (S), Pseudomonas fluorescens (S), Pseudomonas chlororaphis (S), , Silicibacter pomeroyi (S), Rhodobacter sphaeroides KD131 (S), Ruegeria sp. TM1040 (S), Mesorhizobium loti MAFF30399 (S), and Bacillus anthracis (S).

10. The method according to claim 2, wherein the TA enzyme is a mutant ω-transaminase based on the sequence from Arthrobacter sp. or from Bacillus megaterium.

11. A method for obtaining L-glufosinate ammonium salt of formula L-(1) by deracemization of glufosinate-ammonium of formula DL-(1), wherein
in a starting step an oxidative deamination of D-glufosinate to PPO (2-oxo-4-(hydroxy(methyl)phosphinoyl)butyric acid) is carried out with a D-amino acid oxidase (DAAO) enzyme, and
in a following step the PPO is aminated to a L-glufosinate C₁-C₆-alkylammonium salt of formula L-(2) by a transaminase (TA) enzyme using an amine group from an amine donor selected from the group consisting of ethylamine, n-propylamine, isopropylamine, n-butylamine, sec-butylamine, amylamine, sec-pentylamine, n-hexylamine, and sec-hexylamine, 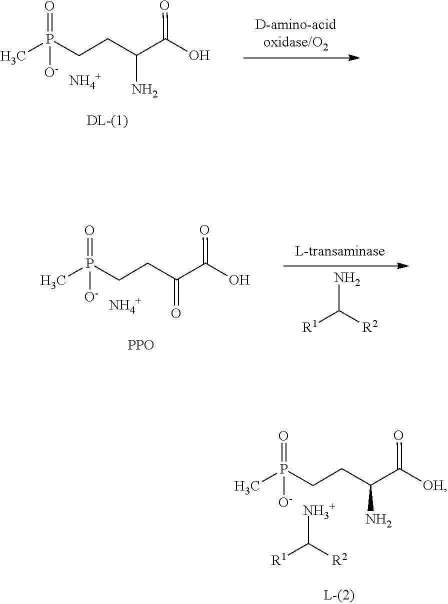
wherein R¹ and R² in formula L-(2) are selected independently from one another from methyl, ethyl, propyl, butyl, or pentyl, and
in a further following step Ca(OH)₂ is added to the aqueous solution of L-glufosinate C₁-C₆-alkylammonium salt of formula L-(2), whereby it reacts to L-glufosinate calcium salt of formula L-(3), and the released low-boiling C₁-C₆-alkylamine is removed by distillation: 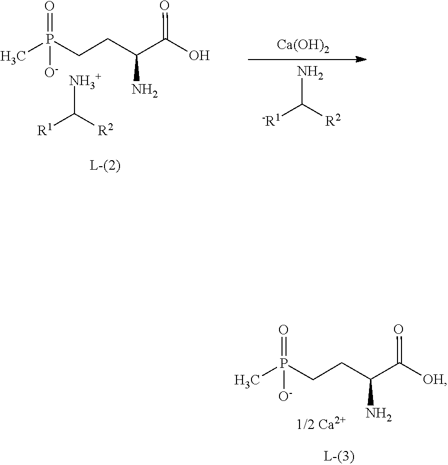
and
in a further following step (NH₄)₂SO₄ is added to the remaining aqueous solution of the calcium salt of L-glufosinate of formula L-(3), whereby the calcium ion of the L-glufosinate calcium salt of formula L-(3) is replaced by the ammonium ion of the ammonium sulfate, and the precipitated calcium sulfate (gypsum) is removed by filtration: 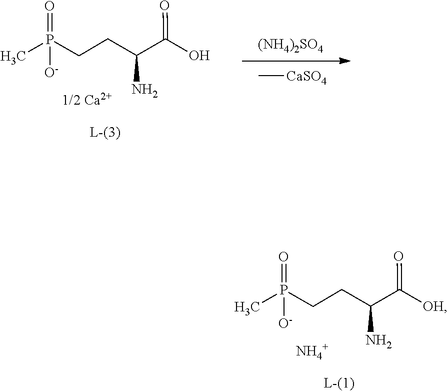
and wherein
in a final step the desired L-glufosinate ammonium salt of formula L-(1) is obtained by removal of water from the clear filtrate.

12. The method of claim 11, wherein the all method steps are performed consecutively as one-pot-process in a single container.

13. The method of claim 11, wherein the individual method steps are performed consecutively as multi-pot-process in separate containers.

14. A method for obtaining an ammonium salt of an organic carboxylic acid RCOONH₄ of formula (6) having an organic moiety R being an (hetero)aromatic or (hetero)aliphatic, (hetero)cyclic or straight/branched open-chained, saturated or unsaturated moiety, optionally comprising one or more heteroatoms,
which method comprises
(A) a first step, wherein an intermediate calcium salt of the carboxylic acid of formula (5) is produced from a C₁-C₆-alkylammonium salt of a carboxylic acid of formula (4), wherein R¹ and R² in formula (4) are selected independently from one another from hydrogen, methyl, ethyl, propyl, butyl, or pentyl;
by adding Ca(OH)₂ and subsequently removing the displaced C₁-C₆-alkylamine by distillation, and wherein
(B) in a second step, the ammonium salt of the carboxylic acid RCOONH₄ of formula (6) is obtained by displacing the calcium ion of the carboxylic acid calcium salt of formula (5) by adding ammonium sulfate and subsequently removing the precipitated gypsum CaSO₄ by filtration:

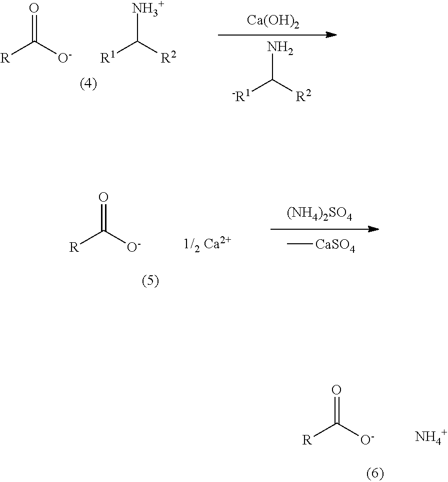

15. A method according to claim 14, wherein the intermediate calcium salt of the carboxylic acid of formula (5) is produced from a secondary C₁-C₆-alkylammonium salt of the carboxylic acid of formula (4), wherein R¹ and R² in formula (4) are selected independently from one another from methyl, ethyl, propyl, butyl, or pentyl.
